(19) United States
(12) Patent Application Publication
(10) Pub. No.: US 2001/0010760 A1
(43) Pub. Date: Aug. 2, 2001

(54) Intraoral imaging camera system

(75) Inventor: Masashi Saito, Kawagoe-shi (JP)

Correspondence Address: Jordan and Hamburg, 122 East 42nd Street, New York, NY, 10168, US

(73) Assignee: Masashi SAITO, Hiroyuki OGUNI, Yoshiteru OKADA and Hiroshi OKADA

(21) Appl. No.: 09/750,605

(22) Filed: Dec. 28, 2000

(30) Foreign Application Priority Data
Jan. 28, 2000 (JP) 2000-20148

(51) Int. Cl. A61B 1/04; G03B 29/00

(52) U.S. Cl. 396/16

(57) ABSTRACT

In a digital camera system comprising lens-barrels 12, 13 having an optical system including an objective 14, a finder 17, a monitoring liquid crystal display 18, a release button 19 and a controller 22 having a power supply, a ring flash 15 is provided on the lens-barrel so as to surround the objective, a metering sensor 26 is provided below the front end portion of the lens-barrel, and a light receiving surface 28 is inclined at an angle of 20 to 85 degrees with respect to the surface which is perpendicular to the optical axis of the optical system. Accordingly, the metering sensor reliably receives the reflected light from a subject, and the amount of light emission of the ring flash can be accurately controlled, so that it is possible to obtain clear images.

### BACKGROUND OF THE INVENTION AND RELATED ART STATEMENT

The present invention relates to an intraoral imaging camera system which enables a dentist or an assistant to take photographs of the interior of an oral cavity and store the photographs as data before straightening irregular teeth of a patient and during the course of straightening in orthodontics.

Conventionally, in orthodontics, before treating teeth of a patient, photographs of the teeth of the patient are taken and stored as data before treatment, and a plan for the treatment is made on the basis of those photographs. In addition, during the course of orthodontic treatment, a dentist or an assistant takes photographs of the interior of an oral cavity to store them as data for enabling both the dentist and the patient to know the progress of the treatment and for use as presentation materials in academic conferences.

As described above, in orthodontics, intraoral imaging of a patient has been frequently conducted, however, with common still cameras, it is impossible to check the taken photographs on the spot whether the photographs have been taken clearly. Furthermore, there has been inconvenience that such photographs as they are cannot be stored as data in a personal computer or the like.

In view of the above and the recent proliferation of digital cameras which enable checking of images on the spot, attempts have been made to conduct intraoral imaging by using digital cameras.

By the way, most of the digital cameras commercially available cannot use a ring flash capable of exposure control of the TTL automatic dimmer type, so that it is inevitable to use a general-purpose ring flash. In such a case, a metering sensor is provided on the top of a camera body, the metering sensor is attached in a position apart from a lens-barrel having an optical system including an objective, and a light receiving surface of the metering sensor is parallel to the optical axis of the lens-barrel.

For this reason, in the case of normal imaging, upon pushing a release button while directing the lens-barrel to a subject such as incisors of a patient, the ring flash emits light, the flash light impinges on the subject, and then the metering sensor receives the light reflected by the subject. Then, a signal from the metering sensor is transmitted to a controller which controls the amount of light emission by the ring flash and stops the light emission of the ring flash.

On the other hand, in the case of special imaging for imaging a small subject such as the interior of an oral cavity of a patient, upon pushing the release button while directing the objective and the ring flash of the camera to the subject, the flash light of the ring flash impinges on the oral cavity of the subject and then the reflected light returns to the camera, however, at the same time, the flash light having passed through the subject and reflected by objects in the vicinity of the subject such as wall and blind is also received by the metering sensor. As a result of this, the controller receiving a signal from the metering sensor cannot control the amount of light emission of the ring flash with accuracy, so that it is impossible to image the intended subject with clarity.

Now, an explanation of intraoral imaging of a patient in dental treatment will be made. As shown in FIG. 4, in the case of mainly imaging incisors of the patient by directing a camera 2 from the front side of a face 1 of the patient who is sitting on the chair on his/her back, there are two cases: the case that the left molars are mainly imaged by directing the camera 2 from the diagonally right side of the face 1 of the patient as shown in FIG. 5, and the second case that the right molars are mainly imaged by directing the camera 2 from the diagonally left side of the face 1 of the patient as shown in FIG. 6.

At this time, as described above, in the conventional digital camera, the metering sensor is provided on the top of the camera body, and this metering sensor is attached in a position apart from a lens-barrel 3 having an optical system including an objective. As a consequence, there arises inconvenience that even if the lens-barrel 3 having the optical system including the objective is directed to the incisors to be imaged as shown in FIG. 4, flash light from the ring flash provided at the front end portion of the lens-barrel 3 passes through the head of the patient to impinge on the wall or floor so that the metering sensor receives the light reflected from the wall or floor.

Moreover, also in the case where the camera is directed to the molars to be imaged as shown in FIGS. 5 and 6, the flash light passes through the face of the patient to impinge on the wall or screen so that the metering sensor receives the reflected light from the wall or screen. Accordingly, it is impossible to obtain clear images in the case of special imaging for imaging a small subject such as intraoral imaging of patient.

### OBJECT AND SUMMARY OF THE INVENTION

It is an object of the invention to provide an intraoral imaging camera system capable of clearly imaging the interior of an oral cavity of a patient principally in orthodontic treatment.

A camera system (including digital camera system) according to the present invention is featured by comprising: a camera body which comprises a lens-barrel having an optical system including an objective, a finder, a monitoring liquid crystal display, a release button and a controller having a power supply; a ring flash provided on the lens-barrel so as to surround the objective; a metering sensor provided at the front end portion of the lens-barrel; and a light receiving surface provided on the metering sensor, wherein the light receiving surface of the metering sensor is inclined at a predetermined angle with respect to the optical axis of the optical system.

The angle of inclination in attaching the light receiving surface of the metering sensor is 20 to 85 degrees with respect to the surface perpendicular to the optical axis of the optical system.

Accordingly, in the invention, the metering sensor receives the reflected light from the subject accurately, so that the amount of light emission of the ring flash can be accurately controlled, and hence clear images can be obtained.

In another feature of the present invention, a ring flash is provided on the lens-barrel via a close-up lens. By providing the close-up lens in this manner, it is possible to realize macro imaging while keeping a sufficient distance with a subject.

In yet another feature of the invention, the metering sensor is rotatably attached in the circumferential direction of the ring flash. In particular, at the time of imaging, the metering sensor is positioned below the ring flash. Accordingly, when the metering sensor is in its free state, the metering sensor is automatically positioned below the ring flash due to its dead weight for allowing imaging. Furthermore, in the case where the digital camera is placed on a table or the like, the metering sensor can be placed horizontally and the digital camera can be placed in a stable condition, with the result that it is possible to protect the metering sensor from shock and prevent it from being damaged. Since appropriate positioning is possible in accordance with needs because the metering sensor freely rotates in the circumferential direction of the ring flash, it is possible to obtain appropriate exposure in accordance with a variety of subjects.

Furthermore, the camera of the present invention is not limited to the digital camera, and also when the invention is applied to a still camera, the metering sensor receives the reflected light from the subject and hence the amount of light emission of the ring flash can be accurately controlled, resulting that it is possible to obtain clear images.

### BRIEF DESCRIPTION OF THE DRAWINGS

FIG. 1 is a perspective view of a digital camera.

FIG. 2 is a rear view of the digital camera.

FIG. 3 is a side view showing an attachment state of a metering sensor.

FIG. 4 is a perspective view showing the manner in which incisors are mainly imaged while directing the camera from the front side of a face of a patient.

FIG. 5 is a perspective view showing the manner in which left molars are mainly imaged while directing the camera from the diagonally right side of the face of the patient.

FIG. 6 is a perspective view showing the manner in which right molars are mainly imaged while directing the camera from the diagonally left side of the face of the patient.

### DETAILED DESCRIPTION OF PREFERRED EMBODIMENTS

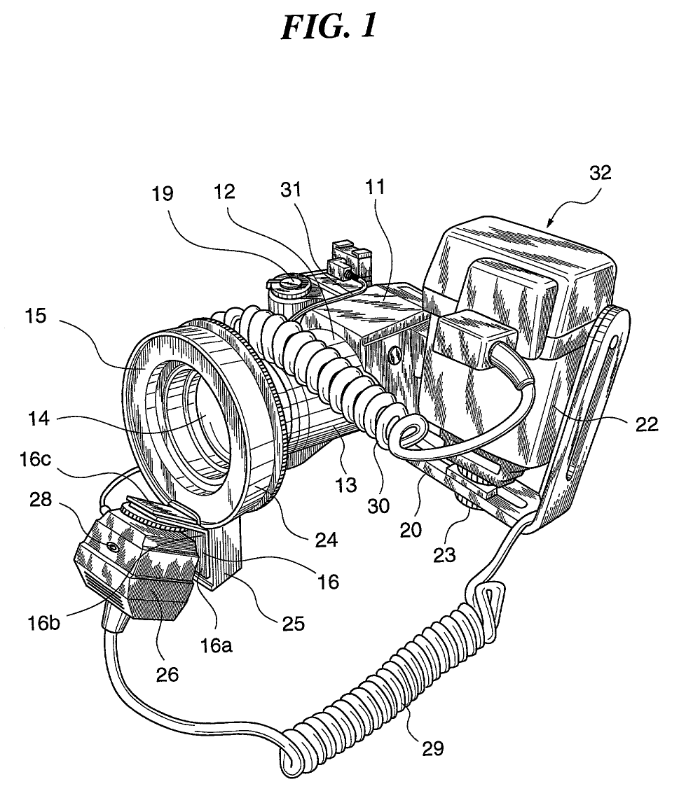

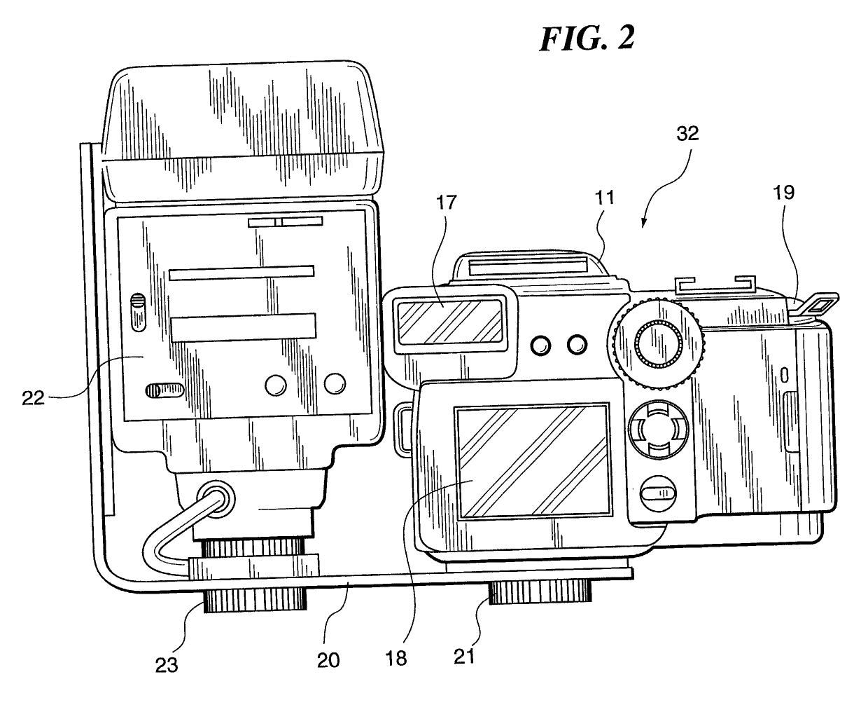

As shown in FIGS. 1 to 3, a camera body 11 of a digital camera 32 is provided with a first lens-barrel 12 having an optical system, and the first lens-barrel 12 is provided with a second lens-barrel 13 so as to be detachable. By attaching a close-up lens having an objective 14 to the second lens-barrel 13, it is possible to realize macro imaging while keeping a sufficient distance with the subject. At the front of the second lens-barrel 13 is provided a ring flash 15 which surrounds the objective 14.

Also the camera body 11 is provided with a finder 17, a monitoring liquid crystal display 18 and a release button 19. A generally L-shaped frame 20 is fixed to the camera body 11 by means of a clamping member 21 such a fixing screw, and a controller 22 having a power supply is fixed to the frame 20 in adjacent to the camera body 11 by means of a clamping member 23 such as a fixing screw. Of course, the controller 22 may be provided integrally with the camera body 11.

In addition, a metering sensor 26 can freely rotate in the circumferential direction of a ring flash 15. Configuration in attaching the metering sensor 26 is as follows: the ring flash 15 is provided with a rotating ring 24 which is rotatable about the optical axis of the objective 14; a sensor adaptor 25 is fixed in a part of the circumference of the rotating ring 24; and the metering sensor 26 is provided on the sensor adapter 25.

The sensor adapter 25 is provided with an attachment piece 16 which is formed by bending a metal piece or a synthetic resin piece. This attachment piece 16 has a proximal end portion 16a which is fixed to the sensor adapter 25, a fold portion 16b provided at its intermediate part, and a distal end portion 16c extending beyond the front of the ring flash 15. And, the metering sensor 26 is attached to the distal end portion 16c of the attachment piece 16 by means of a fixing member 27. Accordingly, the metering sensor 26 is located below the second lens-barrel 13.

The metering sensor 26 has a light receiving surface 28 which receives the reflected light of the flash light emitted toward the subject by the ring flash 15 that has been reflected by the subject and returns to the camera body 11 side. Then, a signal from the metering sensor 26 having sensed the reflected light is transmitted to the controller 22, and the controller controls the amount of light emission of the ring flash 15 and stops the light emission of the ring flash 15.

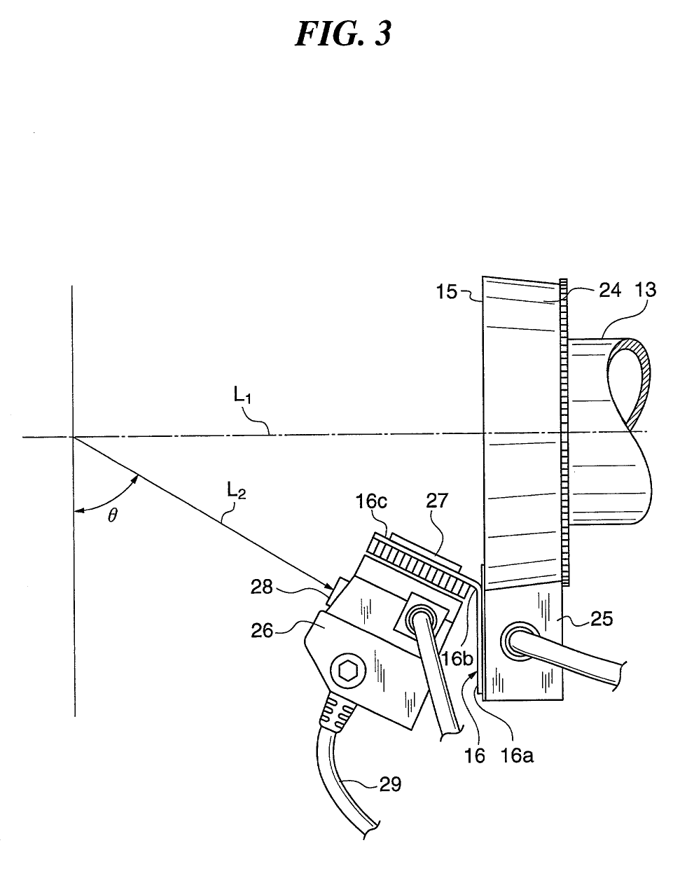

By means of the fold portion 16b of the attachment piece 16, the metering sensor 26 is set so that the light receiving surface 28 of the metering sensor 26 is inclined at an inclination angle θ of 20 to 85 degrees, preferably range of 50 to 70 degrees with respect to the surface which is perpendicular to an optical axis L₁ of the optical system as shown in FIG. 3.

Furthermore, the controller 22 including the power supply and the metering sensor 26 are connected by a first signal cable 29. Furthermore, the controller 22 and the ring flash 15 are connected by a second signal cable 30. The metering sensor 26 and the release button 19 are connected by a third signal cable 31.

According to the digital camera 32 thus constituted, the metering sensor 26 is fixed via the sensor adapter 25 to a part of the rotating ring 24 which can freely rotate with respect to the ring flash 15, and the first signal cable 29 is connected to the metering sensor 26. As a consequence, when the metering sensor 26 is in its free state, the rotating ring 24 rotates due to the dead weights of the metering sensor 26 and the first signal cable 29 so that the metering sensor 26 is positioned below the ring flash 15.

Furthermore, in the case of placing the digital camera 32 on a table or the like, if the metering sensor 26 projects downward from the ring flash 15, it becomes unstable, however, by turning the rotating ring 24 with fingers, it is possible to make the metering sensor 26 horizontal, with the result that the digital camera can be placed in a stable manner and the metering sensor 26 can be protected from shock and prevented from being damaged.

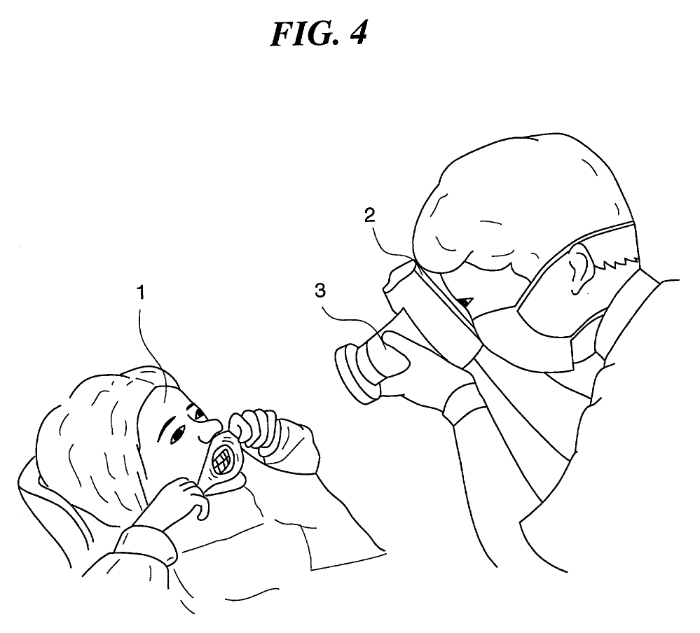

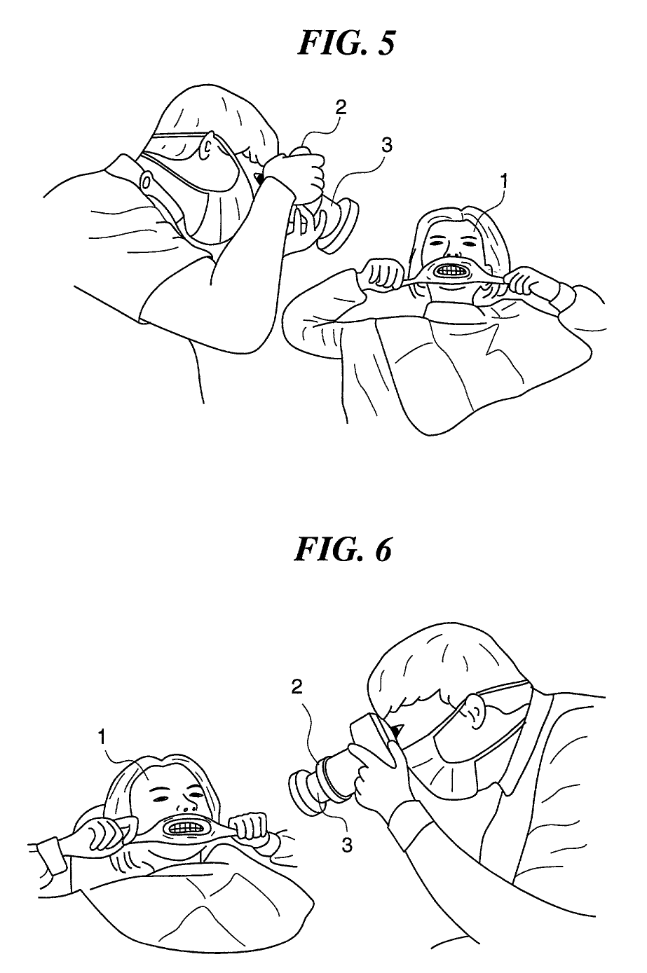

Next, the case of conducting intraoral imaging in dentistry with the use of the digital camera 32 will be described below. In this context, as shown in FIG. 4, the case where the incisors are mainly imaged by directing the digital camera 32 from the front side of the face 1 of the patient who is sitting on the chair on his/her back will be described. In response to that a dentist or an assistant holds the digital camera 32 with his/her left hand, the rotating ring 24 rotates due to the dead weights of the metering sensor 26 and the first signal cable 29 so that the metering sensor 26 is positioned below the ring flash 15.

Looking through the finder 17, an auto-focus frame is adjusted to the incisors (subject) to be imaged from the front side of the face 1 of the patient. Then, upon pressing the release button 19 with the forefinger of the right hand thereon, the ring flash 15 emits light, the light impinges on the incisors, and the reflected light is received by the metering sensor 26. Then, a signal from the metering sensor 26 is transmitted to the controller 22, and the controller 22 controls the amount of light emission of the ring flash 15 and stops the light emission of the ring flash 15.

At this time, since the light receiving surface 28 of the metering sensor 26 is inclined at a predetermined angle with respect to the surface which is perpendicular to the optical axis L₁ of the objective optical system, reflected light L₂ from the incisors reliably enters the light receiving surface 28 of the metering sensor 26, so that the reflected light can be detected by the metering sensor 26 and the amount of light emission of the ring flash 15 is controlled and the light emission of the ring flash 15 is stopped. In addition, an image of the incisors is captured via the optical system including the objective 14 to be formed on a CCD image pickup device, and data of the formed image is converted into a digital signal and the image is stored one by one in a frame memory.

In this case, when conducting intraoral imaging in dentistry, it is not necessary to fill the finder 17 with a single tooth, and alternatively it is also possible to image the entire dentition while breaking down it into the front surface, the side face and the occlusal surface and afterward to select arbitrary portions on the personal computer for processing at pleasure.

As described above, according to the present invention, since the metering sensor reliably receives the reflected light from the subject, and the amount of light emission of the ring flash can be accurately controlled, it is possible to obtain a clear image. In addition, by providing the close-up lens, it is possible to realize macro imaging while keeping a sufficient distance with the subject.

Furthermore, when the metering sensor is in its free state, when the metering sensor 26 is in its free state, the metering sensor is automatically positioned below the ring flash due to its dead weight so that imaging is enabled. Furthermore, in the case of placing the digital camera on a table or the like, it is possible to make the metering sensor horizontal, with the result that the digital camera can be placed in a stable manner and the metering sensor is protected from shock and prevented from being damaged.

Accordingly, in dentistry, especially in orthodontics, the present invention is particularly advantageous in special imaging for imaging a small subject such as intraoral imaging of a patient, and in addition, when applied to a digital camera, the invention make it possible to take photographs of teeth of the patient before treating the teeth, store the photographs as data before treatment, and make a plan for the treatment on the basis of such photographs. In addition, such photographs are useful for both the dentist and the patient to know the progress of the treatment during the course of the treatment, and also such photographs can be utilized as presentation materials in academic conferences if stored as data.

While the explanation of the above-described embodiment has been made with regard to a digital camera, the similar effects can be obtained also in common still cameras by providing the ring flash with the rotating ring capable of freely rotating about the optical axis of the optical system, and attaching the metering sensor in a part of the circumference of the rotating ring via the sensor adapter at an angle.

1. An intraoral imaging camera system comprising:
a camera body which comprises a lens-barrel having an optical system including an objective, a finder, a monitoring liquid crystal display, a release button and a controller having a power supply;
a ring flash provided on the lens-barrel so as to surround the objective;
a metering sensor provided on the lens-barrel; and
a light receiving surface provided on the metering sensor so as to be inclined at a predetermined angle with respect to a surface which is perpendicular to an optical axis of the optical system, for the receiving reflected light of flash light emitted by the ring flash that has been reflected by the subject and returns to the camera body side.

2. The intraoral imaging camera system according to claim 1, wherein the camera is a digital camera.

3. The intraoral imaging camera system according to claim 1, wherein the lens-barrel is provided with a close-up lens having an objective.

4. The intraoral imaging camera system according to claim 1, wherein the metering sensor is provided below the front end portion of the lens-barrel.

5. The intraoral imaging camera system according to claim 1, wherein the controller having the power supply is provided on the camera body via a frame.

6. The intraoral imaging camera system according to claim 1, wherein the light receiving surface provided on the metering sensor is attached so as to be inclined at an inclination angle of 20 to 85 degrees with respect to the surface which is perpendicular to the optical axis of the optical system.

7. The intraoral imaging camera system according to claim 1, wherein the metering sensor is rotatably attached in the circumferential direction of the ring flash.

8. The intraoral imaging camera system according to claim 1, wherein the ring flash is provided with a rotating ring which is capable of rotating about the optical axis of the objective, a sensor adapter is fixed to the rotating ring, and the metering sensor is provided on the sensor adapter.

9. The intraoral imaging camera system according to claim 1, wherein the ring flash is provided with a rotating ring which is capable of rotating about the optical axis of the objective, a sensor adapter is fixed to the rotating ring, and the metering sensor is provided on the sensor adapter via an attachment piece having a fold portion.

10. The intraoral imaging camera system according to claim 1, wherein the metering sensor rotatably provided on the ring flash and the controller are connected by a signal cable.

11. The intraoral imaging camera system according to claim 1, wherein the metering sensor is positioned below the ring flash during imaging.
